(19) United States
(12) Patent Application Publication
(10) Pub. No.: US 2005/0070464 A1
(43) Pub. Date: Mar. 31, 2005

(54) Sialic carbohydrates

(76) Inventors: Bernd Stahl, Rosbach (DE); Sorge Kelm, Lilienthal (DE); Gunther Boehm, Echzell (DE); Berndt Finke, Herford (DE); Joachim Schmitt, Hosbach (DE)

Correspondence Address: BACON & THOMAS, PLLC, 625 SLATERS LANE, FOURTH FLOOR, ALEXANDRIA, VA, 22314, US

(21) Appl. No.: 10/502,049

(22) PCT Filed: Jan. 31, 2003
(86) PCT No.: PCT/EP03/00980

(30) Foreign Application Priority Data
Feb. 1, 2002 (DE) 102 04 000.1

(51) Int. Cl. A61K 38/16; A61K 31/739

(52) U.S. Cl. 514/8; 514/54

(57) ABSTRACT

The invention relates to the use of sialic carbohydrates of general formula (I), containing at least one carbohydrate unit of general formula (II) for the immune modulation, immune suppression and in the prevention and treatment of infections in humans and animals. In said formula (II), Sia represents a sialic acid or a sialic acid derivative in an α2-3 bond, Gal represents a galactose-monosaccharide-unit, HexNac represents an N-acetylated galactosamine or glucosamine-monosaccharide-unit (GalNAc or GlcNAc), Hex represents a galactose- or glucose-monosaccharide-unit (Gal or Glc), C represents HexNac or Hex or is absent, n represents 1-50, V represents OH, a carbohydrate radical or a connecting point on a support T, with the proviso that if V represents OH, n represents 1, and if V represents a carbohydrate radical or a support T, n represents the number of carbohydrate unites of general formula (II) directly connected to said carbohydrate radical or support, X represents a sialic acid or a sialic acid derivative, wherein a second sialic acid or a sialic acid derivative or more sialic acid or sialic acid derivatives can be connected to an α2-8 bond, a phosphate, a sulphate, or carboxyl-group or a monosaccharide having a phosphate, sulphate, or carboxyl group and only one of the radicals X is present.

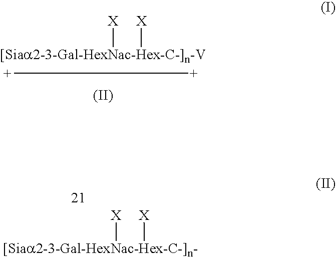

The invention relates to the use of carbohydrates for immunomodulation, immunosuppression and for treating infections in humans and animals, and to food, dietetic and pharmaceutical compositions containing these carbohydrates.

The adhesion of pathogenic organisms, as well as of cell-damaging substances to the surface of mammal cells is the first step and an essential prerequisite for an infection or a damage of the cell. The interaction between the pathogens and the cells is formed by a ligand-receptor relationship. In this ligand-receptor relationships or interactions, glycosidic structures play an important role.

One possibility of influencing such ligand-receptor relationships consists in blocking the respective receptors or ligands, respectively, of one of the two cell surfaces or of both cell surfaces.

Using specific test systems, it could be shown that various carbohydrate mixtures reduce or even completely prevent the adhesion of, for example, micro-organisms to the cell surface, cf.: Kunz, C; Rudloff, S. Acta Paediatr. 1993, 82, 903-912. Other substances such as the Lewis structures as the carbohydrate ligands of selectines (adhesion proteins on endothelial cells and lymphocytes) modulate the interaction of lymphocytes with the endothelium, for example, within the scope of rolling, homing and the invasion during inflammatory processes (Albelda S. M., Smith C. W., Ward P. A., FASEB J. 1994, 8, 504-512). The group of sialic acids yields an important contribution in the above-mentioned pathogen-cell interactions on the one hand and in cell-cell interactions on the other hand. These monosaccharides deploy their action in particular as components of oligosaccharides and glycoconjugates such as glycolipids and glycoproteins (Traving and Schauer, Structure, function and metabolism of sialic acids, Cell. Mol. Life Sci., Vol. 54, 1998). Sialic acids are to be found in up to 40 different derivatization forms, and have hitherto been detected in humans, animals and some viruses, bacteria and fungi.

Apart from the chemico-physical effect of the negative charge of the carboxyl group, the main function of sialic acids is their specific participation in molecular and cellular recognition processes (Kelm S. & Schauer R. (1997), Sialic acids in molecular and cellular interactions, Int. Rev. Cytol. 175, 137-240). A part thereof is the stabilization of enzymes and other proteins, autorecognition/non-autorecognition of the immune system, masking of cells and proteins. Siglecs (P. R. Crocker et al. (1998) Siglecs—a family of sialic acid-binding lectins, Glycobiol., 8, Glycoforum 2 v-vi.) constitute a particular class of sialic acid-specific lectins. These molecules have immunoglobulin-like domains, represent lectin-like receptors, and, as an essential feature, bind sialic acid.

In cell-cell interactions, the structure of the ligands or receptors is generally essential for the action mechanism of these interactions. The binding process of a ligand to a receptor is the first event that may lead to a triggering of further signal cascades, Also in the case of siglecs, it could be shown that these participate in signal transduction mechanisms. Thus, tyrosines, which are for example present in the Siglec-2 (CD22) may undergo a phosphorylation, which in turn, and through further intermediate steps, leads to a reduced activation of B-cells.

The binding mechanism of specific proteins to carbohydrate structures is also of importance for the adhesion of bacteria to epithelial surfaces. Since the adhesion represents the first step of a first infection, the inhibition of this binding process is an important objective in avoiding infections.

The bacterial lectins (adhesins) specifically recognizing α2-3 are subject to the same mechanism that applies also to the siglecs.

Moreover, what is known as clustering both of receptors and ligands plays an important role in the binding intensity. An important criterion is in this case the polyvalence of the binding-active ligands. The more ligand structures are combined in a molecule, the more pronounced the interactions between ligands and receptors or between two or more cells may be. This applies in particular for interactions where a cross-linkage of molecules or cells modulates the function of a biological process. It thus has to be emphasized that polyvalent ligands lead to a cross-linkage of receptors and hence may provoke another signal effect.

The object of the present invention is to show a way how an immunomodulation, immunosuppression and a treatment of infections in humans and animals may be achieved with the help of carbohydrates.

This object is solved by the teachings of the claims.

According to the invention, the sialyzed carbohydrates of the following general formula I having at least one carbohydrate unit of the following general formula II 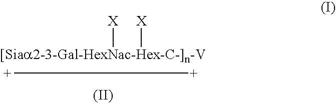
wherein
Sia means a sialic acid or a sialic acid derivative in an α2-3 bond,
Gal means a galactose-monosaccharide unit,
HexNac means an N-acetylated galactosamine-monosaccharide unit or glucosamine-monosaccharide unit (GalNAc or GlcNAc),
Hex means a galactose-monosaccharide unit or glucose-monosaccharide unit (Gal or Glc),
C represents HexNac or Hex or is absent,
n represents 1 to 50,
V represents OH, a carbohydrate residue or a connecting point on a carrier T, with the proviso that if V represents OH, n represents 1, and, if V represents a carbohydrate residue or a carrier T, n means the number of the carbohydrate units that are each directly bound to this carbohydrate residue or carrier and which are of the general formula II 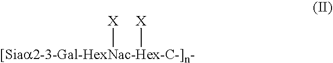
X means a sialic acid or a sialic acid derivative thereof, wherein a second sialic acid or a sialic acid derivative or several sialic acids or sialic acid derivatives can be bound to the sialic acid or the sialic acid derivative in an α2-8 bond, a phosphate group, sulphate group or carboxyl group, or a monosaccharide including a phosphate group, sulphate group or carboxyl group, and only one of the residues X is present,
are used for the immunomodulation, immunosuppression and prevention as well as treatment of infections in humans and animals.

Surprisingly, it has been found that rather than the initially mentioned oligosaccharide sequences, the inventively used carbohydrates are highly efficient inhibitors or receptor analogues. These carbohydrates will hereinafter be called “inventive carbohydrates”.

The inventive carbohydrates must feature at least one carbohydrate unit of the general formula II [Siaα2-3-Gal-HexNac(X)-Hex(X)—C-]-; in this case, the residues Sia, Gal HexNac, Hex, X and C have the meanings indicated in conjunction with general formula I.

This carbohydrate unit of the general formula II thus constitutes a component of the inventive carbohydrates of general formula I. If the residue V in the formula of general formula I represents OH, then the carbohydrates of general formula I are made up of a carbohydrate unit of the general formula II and this residue V.

In the sialyzed carbohydrates, the preferred linkages are the following: α1-2, α1-3, α1-4, α1-6, α2-3, α2-6, α2-8, β1-2, β1-3, β1-4, and β1-6. In a carbohydrate or glycan, one as well as several of the mentioned bonds may occur. Preferred are β-linked base structures (I, II) with charged a-glycosidic groups, insofar as X also stands for a glycan.

The residue V may also represent a carbohydrate residue or a linkage point on a carrier T. In the latter case, V, strictly speaking, does not mean a residue but just this linkage point. Via this linkage point, the carbohydrate unit of the general formula II is bound to a carrier or is immobilized thereon. The kind of carrier is not particularly critical so that any one of a large number of carriers may be used. Up to 50 carbohydrate units of general formula II may be bound to such a carrier, each of which carbohydrate units is bound or coupled to this carrier T through the linkage point V. The index n used within the framework of the present documents thus stands for the number of carbohydrate units of the general formula II, and not, for instance, a chain of n-elements of this carbohydrate units.

Where it is stated that n represents 1-50, then this means that n represents an integer from 1 to 50, and hence may represent 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50.

The carrier T is in particular a peptide, a protein, a polymer or a biopolymer, with the linkage with the peptide or protein in particular being N-glycosidic or O-glycosidic. Polyhydroxybutanoic acid may be used as biopolymer. A suitable polymer is, for example, polyacrylamide.

The residue V may also represent a carbohydrate residue. This is preferably a monosaccharide residue, oligosaccharide residue or polysaccharide residue. Examples may be the following: milk oligosaccharides, reserve carbohydrates (in particular starch) and skeletal carbohydrates such as pectins, celluloses and galactomannans. While it is true that these carbohydrates may also be called biopolymers, for a better terminological delimitation they will not be subsumed under the above-mentioned biopolymers for the purposes of the present invention.

The carbohydrate residue representing the residue V may also be one or more (even aliphatic) carbohydrate residue(s) composed like the carbohydrate units of general formula II, with the bonds of these carbohydrate residues being different.

It is crucial that the carbohydrate unit of general formula II has at least two negative charges present on a nucleus of at least 3 and in particular 4 neutral monosaccharides (Gal, HexNac, Hex and C). One of these negative charges is located on the non-reducing end of the nucleus (it may also be designated as core) as an α2-3 linked sialic acid on a galactose. The second charge is also present in a subterminal location as a sialic acid, which, however, may be substituted by other charged groups, i.e. phosphate, sulphate or carboxyl groups. These phosphate, sulphate and carboxyl groups may be located directly at the monosaccharides HexNac and Hex or may be bound to another monosaccharide. In other words, this other monosaccharide will then represent the bridge between this charged group and the monosaccharide HexNac or Hex of the carbohydrate unit of general formula II.

If the residue V is a carbohydrate residue corresponding to the carbohydrate unit of general formula II as far as its composition is concerned, then preferably those residues V are used in which two negative charges per 3 or 4 neutral monosaccharides are present.

In the carbohydrate units of general formula II, only one of the residues X is present. In other words, it is either the monosaccharide HexNac or the monosaccharide Hex that carries a residue X of the above mentioned kind.

If monosaccharides are mentioned within the framework of the present documents for explaining the general formulae I and II, then, strictly speaking, the term “monosaccharide unit” would have to be used, since these monosaccharides are not present in an isolated form but are bound to other monosaccharides or groups. For the sake of simplicity, however, only the term monosaccharide or the specific name of the monosaccharide (e.g. galactose, glucose, hexosamine, etc.) will be used.

The monosaccharide unit designated Sia preferably is acetyl neuraminic acid (NeuAc) or N-glycolyl neuraminic acid (NeuGc). The O-acyl derivative of the sialic acid of the residues Sia and X, for example, is an O-acetyl derivative.

If the residue X signifies a sialic acid or an O-acyl derivative, then it is preferably NeuAc, NeuGc and the derivatives thereof, e.g. O-acetylated sialic acid in an α2-3 bond or an a2-6 bond.

As already explained above, a second sialic acid or several sialic acids may be present on the residue X in an α2-8 bond.

While the α2-3 bound Neu5Ac (residue Siaα2-3 in the general formulae I and II), in order to produce a bond, absolutely must be present in the carbohydrates used according to the invention as a necessary recognition sequence, the second and third Neu5Ac are only necessary because of the charge in order to increase the affinity. The kind of bond is then not important. This second Neu5Ac (residue X) may also be substituted by another group carrying a negative charge (phosphate, sulphate, carboxyl, also in the form of carboxylate).

For the immunomodulation, immunosuppression and the treatment of infections in humans and animals with the help of the inventive carbohydrates, primarily the carbohydrate units of general formula II are thus of importance. The kind of the carrier T to which these carbohydrate units of general formula II may be bound, is therefore of a subordinate importance. The carrier T may therefore be of any desired kind. Preferably, it is a carbohydrate, a peptide, a protein, a polymer or a biopolymer, with the linkage with the peptide or protein preferably being N-glycosidic or O-glycosidic. More preferably, the carrier T consists of lipophilic compounds including one or more carbohydrate unit(s) of general formula II which represent(s) the head group(s) thereof. Preferred lipophilic compounds are glycolipids and gangliosides.

As the carbohydrate of general formula I, disialyl-lacto-N-tetraose (DS-LNT), disialyl-lacto-N-neo-tetraose (DS-LNnT), glycomacropeptide (GMP) and the gangliosides G_{D1a}, G_{T1b} and G_{T1c} are used. The GMP preferably originates from animal milks such as sheep, goat, buffalo, camel and in particular cow milk.

The carbohydrates used according to the invention, be they free carbohydrates (V stands for H₂O) or carbohydrates bound to a carbohydrate residue or to a carrier T, may or will be incorporated into various food, dietetic and pharmaceutical compositions. All of these compositions may be present in a fluid or solid form. The term “food composition” used herein not only comprises the actual food composition but also food additives, food supplements, beverages and food compositions including infant and baby formulae. The term “baby or infant formulae” refers in particular to all artificially prepared formulae, in particular for human babies but no human milk. “Artificial” means those infant formulae which are produced from vegetable or animal raw materials but not from human origin. These food compositions may be administered to a human being or an animal in any desired way. This also includes administration into the stomach as a probe food.

The carbohydrates according to the invention may, for example, be added as admixtures or additives to the following products, although this enumeration is not conclusive: milk and milk products, infant and babyfood formulations, chocolate bars, yoghurt drinks, cheese, sausage and meat products, anabolic food, probe food and products for pregnant women.

The carbohydrates according to the invention may also be administered in the form of a pharmaceutical composition alone or together with one or several additional active agent(s). These compositions may, for example, be formulated as a tablet/sachet. For the formulation of such pharmaceuticals, usual adjuvants, carriers, auxiliary agents, diluents, moisturizing agents, thickening agents, flavoring agents, sweetening agents, etc. may be used.

The pharmaceutical compositions may be administered in any usual way to a patient (i.e. human and animal). However, for the sake of convenience, they will be compositions suited for oral, lingual, nasal, intestinal, bronchial, vaginal, topical (skin and mucosa) and per os administration and formulated to suit the kind of administration.

The foods, dietetic compositions and pharmaceutical compositions containing at least one carbohydrate used according to the invention, may be used among other things for preventing and treating infections of the gastrointestinal tract, e.g. in case of listerioses, of the blood system, the respiratory passages, the urogenital tract, as well as of the nasopharynx, and for protecting endothelia, epithelia and mucosa. Thus, they may be applied topically to the skin or may also be used on mucous membranes. These mucous membranes include nasal, intestinal, bronchial and vaginal mucous membranes. Thus, the carbohydrates used according to the invention may, for example, be added to a mouthwash. All age groups, ranging from new born babies up to elderly people, may be mentioned as target groups for the carbohydrates used according to the invention. Special fields of application are the protection and the treatment of pregnant women, sick persons, debilitated and elderly people, for whom the prevention e.g. of a listeriosis is of particular importance.

Due to their anti-adhesive properties, the inventive carbohydrates can thus be used for reducing or preventing an infection. On the other hand, they can also serve the purpose of influencing the immunological response by modulating cell-cell interactions. This applies both to the modulation of cell-cell interactions preferably between eucaryontic cells (e.g. lymphocytes and endothelial cells, etc.) and for the modulation of the adhesion of pathogens (such as bacteria, spores, viruses, viroids, prions, fungi, monocellular and multicellular parasites, toxins and heavy metal cations) on eucaryontic cells, preferably mammalian cells.

The carbohydrates used according to the invention are known compounds, e.g. DS-LNT, DS-LNnT and the gangliosides G_{D1a}, G_{T1b} and G_{T1c}. They are known and/or may be prepared according to suitable known methods in a chemical or enzymatic way, or in a combination of these two technologies.

The disialyl-lacto-N-tetraose (DS-LNT) and disialyl-lacto-N-neo-tetraose (DS-LNnT) used according to the invention are compounds of general formula I, in which HexNac=GlcNac, Hex=galactose and C=glucose. In addition, two sialic acids are present (Siaα2-3NeuAc, X=α2-6NeuAc). Also the derivatives thereof may be used, which are preferably those where the non-terminal sialic acid has been substituted by a glycolyl neuraminic acid, a sulphate group, a phosphate group or a carboxyl group or carboxylat group. A further sialic acid may also belinked. The type 1 lactosamin unit may be replaced by a Gal (β1-3) GalNAc unit.

In the gangliosides G_{D1a}, and G_{T1b} which are further used according to the invention, the monosaccharide sequence is very similar to DS-LNT. The bonding of the second or third sialic acid, however, takes place on the galactose localized on the glucose (junction to the ceramide).

The carbohydrates of general formula I used according to the invention appropriately are administered in such an amount, that a patient preferably is supplied once daily with at least 1 mg of carbohydrate of general formula I, and in particular one carbohydrate unit of general formula II per kg of body weight.

Exemplary dietetics and pharmaceuticals are listed below which contain at least one inventive carbohydrate. These are the following inventive carbohydrates: disialyl-lacto-N-tetraose (DS-LNT) and the derivatives thereof (see above), DS-LNnT and the derivatives thereof, and G_{D1a}, G_{T1b} and G_{T1c}. For the sake of simplicity, these carbohydrates will simply be termed “inventive carbohydrate” in the examples. This term is representative for each of the above-mentioned inventive carbohydrates and the mixtures thereof.

### EXAMPLE 1

For preparing sachets, in each case 100 mg or only 5 mg of an inventive carbohydrate are mixed in a dry state with 990 mg of maltodextrin, and then are packed in sachets. These sachets are administered three times per day during meals.

### EXAMPLE 2

A known medicinal food (i.e. Milupa® HN 25, balanced diet) in the form of a bead product containing 18.8 g of protein, 8.6 g of fat, 62.8 g of carbohydrates, 3.3 g of minerals and vitamins, is admixed in a composition known per se with an inventive carbohydrate in such an amount that 50 mg of the inventive carbohydrate are contained in 100 g of the finished bead product.

For the composition of a liquid medicinal food, 100 ml of the known medicinal food Milupa HN 25 liquid (2.3 g of protein, 1.6 g of fat, 8.5 g of carbohydrates, 37 g of minerals and vitamins) are admixed with 7 mg of an inventive carbohydrate.

### EXAMPLE 3

A product for Pregnant Women

An effervescent tablet (final weight 4.15 g) (Neovin® from Milupa) is prepared in a manner known per se by admixing 200 to 500 mg of an inventive carbohydrate. One tablet per day is dissolved in 150 ml water and swallowed.

### EXAMPLE 4

A Product for the Elderly and Debilitated Persons

A balanced pulverized medicinal food (Dilsana® from Milupa) containing 22.5 g of protein, 7.7 g of fat, 60.8 g of carbohydrates, 5.4 g of minerals and vitamins is prepared in a manner known per se by incorporating 100 mg to 1000 mg of an inventive carbohydrate per 100 g of powder. Up to 3×50 g per day of the food are dissolved in 150 ml water and administered.

### EXAMPLE 5

Tea

100 g of an instant tea powder prepared in the usual manner are mixed with 2 g of an inventive carbohydrate. 3.8 g of tea powder are dissolved in 100 ml of hot water, and administered three times per day.

### EXAMPLE 6

A protein-adapted infant milk formulation (Aptamil® from Milupa) containing 11.8 g of protein, 56.9 g of carbohydrates, 24.9 g of fat, 2.5 g of minerals and vitamins and 45 mg of taurine are prepared in the usual manner in the form of a bead product, which is mixed with 100 mg to 1000 mg of an inventive carbohydrate per 100 g of infant milk formulation.

1. Use of sialyzed carbohydrates of the following general formula I having at least one carbohydrate unit of the following general formula II: 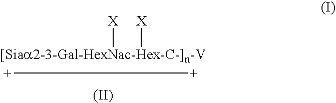
wherein
Sia means a sialic acid or a sialic acid derivative in an α2-3 bond,
Gal means a galactose-monosaccharide unit,
HexNac means an N-acetylated galactosamine-monosaccharide unit or glucosamine-monosaccharide unit (GalNAc or GlcNAc),
Hex means a galactose-monosaccharide unit or glucose-monosaccharide unit (Gal or Glc),
C represents HexNac or Hex or is absent,
n represents 1 to 50,
V represents OH, a carbohydrate residue or a connecting point on a carrier T, with the proviso that, if V represents OH, n represents 1, and, if V represents a carbohydrate residue or a carrier T, n means the number of the carbohydrate units that are each directly bound to this carbohydrate residue or carrier and which are of the general formula II 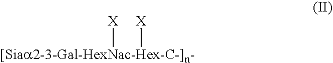
X means a sialic acid or a sialic acid derivative thereof, wherein a second sialic acid or a sialic acid derivative or several sialic acids or sialic acid derivatives can be bound to the sialic acid or the sialic acid derivative in an α2-8 bond, a phosphate group, sulphate group or carboxyl group, or a monosaccharide including a phosphate group, sulphate group or carboxyl group, and only one of the residues X is present,
for the immunomodulation, immunosuppression and prevention as well as treatment of infections in humans and animals.

2. Use according to claim 1, characterized in that one, two, three, four, or all of the following criteria i) through iv) are met:
i) Sia represents acetyl neuraminic acid (NeuAc) or N-glycolyl neuraminic acid (NeuGc),
ii) the sialic acid derivative or the sialic acid derivatives of the residues Sia and X is/are an O-acyl derivative and in particular an O-acetyl derivative,
iii) the carrier T is a peptide, a protein, a polymer or a biopolymer, with the linkage with said peptide or protein in particular being N-glycosidic or O-glycosidic, and
iv) the carbohydrate residue constituting the residue V is a monosaccharide residue, an oligosaccharide residue or a polysaccharide residue.

3. Use according to claim 1, characterized in that the carbohydrates of general formula I are selected from disialyl-lacto-N-tetraose (DS-LNT, V=OH, HexNac=GlcNAc, Hex=galactose (Gal), C=glucose, Sia=α2-3 NeuAc, X=α2-6 NeuAc on HexNac), disialyl-lacto-N-neo-tetraose (DS-LNnT), glycomacropeptide (GMP), ganglioside G_{D1a}, ganglioside G_{T1b} and ganglioside G_{T1c}.

4. Use according to claim 1 any one of claims 1, characterized in that T represents lipophilic compounds, and the carbohydrate unit or carbohydrate units of general formula II represents or represent the head group(s) thereof.

5. Use according to claim 4, characterized in that said lipophilic compounds are glycolipids and in particular gangliosides.

6. Use according to claim 1, characterized in that the carbohydrate or carbohydrates of general formula I is/are used in an amount of at least 1 mg per kg of body weight.

7. Use according to claim 1, for the prevention and treatment of infections of the gastrointestinal tract, blood system, respiratory passages, urogenital tract, as well as the nasopharynx.

8. Use according to claim 1, characterized in that the carbohydrate or carbohydrates of general formula I are incorporated into a fluid or solid food composition (with the exception of human milk), dietetic composition or pharmaceutical composition for administration to a human or an animal, or serve for the preparation of such a composition for the immunomodulation, immunosuppression and treatment of infections in humans and animals.

9. Use according to claim 8, characterized in that the pharmaceutical composition serves for an oral, lingual, nasal, bronchial, vaginal, topical (skin and mucosa) and per os administration, for an administration by means of a probe into the stomach of a human or an animal, or for an administration as an infusion.

10. Food composition, dietetic composition or pharmaceutical composition containing at least one carbohydrate of general formula I as described in claim 1.

11. Composition according to claim 10, characterized in that the composition may contain a further carbohydrate or several further carbohydrates, which are different from the carbohydrates of claim 1, a further active agent or several further active agents and/or a further ingredient, which is known and suited for the corresponding composition, or more of such ingredients, wherein in the case of a pharmaceutical composition a usual auxiliary agent or several usual auxiliary agents, including diluents, moisturizing agents, thickening agents, flavoring agents, sweetening agents and carriers, may be present, and in the case of a food composition or a dietetic composition, at least one further food component may be present.

12. Method of immunomodulation, immunosuppression and treatment of infections in humans and animals, characterized in that at least one carbohydrate of general formula I or a composition according to claim 10 is administered to a human or an animal, but not in the form of human milk, in particular in such an amount that at least 1 mg of carbohydrate of general formula I is administered per kg of body weight to the human or the animal once daily.

13. Sialyzed carbohydrates of the following general formula I having at least one carbohydrate unit of the following general formula II: 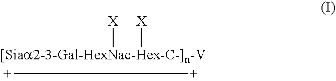
wherein
Sia means a sialic acid or a sialic acid derivative in an α2-3 bond,
Gal means a galactose-monosaccharide unit,
HexNac means an N-acetylated galactosamine-monosaccharide unit or glucosamine-monosaccharide unit (GalNAc or GlcNAc),
Hex means a galactose-monosaccharide unit or glucose-monosaccharide unit (Gal or Glc),
C represents HexNac or Hex or is absent,
n represents 1 to 50,
V represents OH, a carbohydrate residue or a connecting point on a carrier T, with the proviso that, if V represents OH, n represents 1, and, if V represents a carbohydrate residue or a carrier T, n means the number of the carbohydrate units that are each directly bound to this carbohydrate residue or carrier and which are of the general formula II 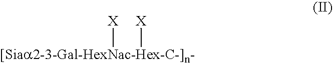
X means a sialic acid or a sialic acid derivative thereof, wherein a second sialic acid or a sialic acid derivative or several sialic acids or sialic acid derivatives can be bound to the sialic acid or the sialic acid derivative in an α2-8 bond, a phosphate group, sulphate group or carboxyl group, or a monosaccharide including a phosphate group, sulphate group or carboxyl group, and only one of the residues X is present,
for the immunomodulation, immunosuppression and prevention as well as treatment of infections in humans and animals.
